(19) United States
(12) Patent Application Publication
(10) Pub. No.: US 2015/0005686 A1
(43) Pub. Date: Jan. 1, 2015

(54) Knee Orthosis Device and Associated Methods

(71) Applicant: University of Connecticut, Farmington, CT (US)

(72) Inventors: Kazem Kazerounian, Storrs, CT (US); Martin Huber, Vernon, CT (US); Horea Ilies, Glastonbury, CT (US); Matthew Eschbach, Wethersfield, CT (US)

(73) Assignee: UNIVERSITY OF CONNECTICUT, Farmington, CT (US)

(21) Appl. No.: 14/319,149

(22) Filed: Jun. 30, 2014

Related U.S. Application Data
(60) Provisional application No. 61/841,488, filed on Jul. 1, 2013.

(51) Int. Cl. A61F 5/01

(52) U.S. Cl. CPC A61F 5/0125
USPC 602/16

(57) ABSTRACT

Exemplary embodiments of a knee orthosis device for providing support to a patient during a gait cycle are provided that generally include a first support mechanism and a second support mechanism. The knee orthosis device generally includes a central mechanism mechanically connecting the first support mechanism and the second support mechanism. A link of the central mechanism can translate relative to the second support mechanism during the gait cycle. The second support mechanism can include a resistive element configured and dimensioned to receive at least a portion of the link therein to provide support to the patient. Exemplary embodiments of a method for providing support to a patient during a gait cycle are also provided.

### CROSS-REFERENCE TO RELATED APPLICATIONS

This application claims the benefit of co-pending, commonly assigned U.S. Provisional Patent Application No. 61/841,488, which was filed on Jul. 1, 2013. The entire content of the foregoing provisional patent application is incorporated herein by reference.

### TECHNICAL FIELD

The present disclosure relates to a knee orthosis device and, more particularly, to a quasi-passive robotic knee orthosis device for reducing loads, moments, or both, in a knee joint of a patient and for providing muscle support to the patient.

### BACKGROUND

As is known in the industry, the gait cycle of a person generally begins with the forward limb making contact with the ground and ends when that limb contacts the ground a second time. After initial contact, the contact limb is said to be in the “loading response phase” and can be referred to as the stance limb. The loading response phase can be characterized by knee flexion (due to the newly acquired load) which must be opposed by the quadriceps extensor muscles. In people with osteoarthritis, weakness in the extensor muscles generally contributes to a significant decrease in the initial shock absorption capabilities of the knee which, in turn, increases the possibility of joint pain.

The midstance phase can begin as the limb opposing the stance limb undergoes toe off (e.g., only the stance limb is in contact with the ground) and ends as the body's center of gravity is directly over the foot. During the midstance phase, the knee must recover from shock absorption of initial contact by extending to support the body as it moves forward.

The next phase can be referred to as the terminal stance phase. The terminal stance phase ends when the contralateral limb makes its initial contact with the ground. During the terminal stance phase, the ground reaction force vector passes in front of the knee and reduces the demand on the thigh muscle. After the terminal stance phase, the limb enters a swing phase, a phase in which the limb does not have any contact with the ground and all support is provided by the contralateral limb. (See, e.g., Perry, J. et al., Gait Analysis: Normal and Pathological Function, 2nd ed., Thorofare, N.J., SLACK Incorporated (2010)).

At points during the loading response and the midstance phase, the knee can encounter loads higher than the total body weight, as well as significant demands on the quadriceps extensor muscles. Reduction of these loads and moments generally lowers pain experienced in the knees of those suffering from osteoarthritis and may provide a favorable environment for joint repair. (See, e.g., Waller, C. et al., Unload it: the key to the treatment of knee osteoarthritis, Knee Surg. Sports Traumatol. Arthrosc., Vol. 19, No. 11, pp. 1823-1829 (November 2011)).

Solutions generally available in the industry include, for example, powered mobility devices, such as mobility scooters and powered wheelchairs, which completely remove any weight being put on the knee. However, these devices simultaneously restrict the overall mobility and independence of the user. As an alternative, a passive custom-fit knee brace can be used to treat osteoarthritis by shifting the weight from the afflicted side of the knee to the healthy side. However, this solution may not be satisfactory when the condition affects both sides of the knee. Surgical solutions are also common, generally in advanced cases of osteoarthritis. The surgical solutions can range from arthroscopic procedures that remove debris in the knee to total joint replacement. In addition, the pharmaceutical industry produces drugs that may control pain, decrease inflammation and/or lubricate joints with the goal of reducing the suffering of the user from the pain caused by osteoarthritis.

Thus, a need exists for a quasi-passive robotic knee orthosis device for reducing loads, moments, or both, in a knee joint of a patient, while allowing the patient to remain mobile and providing muscle support. In addition, a need exists for a quasi-passive robotic knee orthosis device which can be adjusted or customized to provide a variable amount of support to the patient. These and other needs are addressed by the devices and associated methods of the present disclosure.

### SUMMARY

In accordance with embodiments of the present disclosure, exemplary knee orthosis devices for providing support to a patient during a gait cycle are provided. The knee orthosis devices include a first support mechanism and a second support mechanism. The first support mechanism can be adapted to be positioned above a knee joint of the patient. The second support mechanism can be adapted to be positioned below the knee joint of the patient. The knee orthosis devices include a central mechanism adapted to be positioned at or near the knee joint of the patient. The central mechanism can mechanically connect the first support mechanism and the second support mechanism. A link of the central mechanism can translate relative to the second support mechanism during the gait cycle. The second support mechanism can include a resistive element configured and dimensioned to receive at least a portion of the link therein. Interaction between the resistive element and the link provides support to the patient during the gait cycle.

In some embodiments, the central mechanism can be a four-bar linkage system including four links rotatably connected relative to each other. Motion of the four links of the central mechanism can be adjustable based on, e.g., a patient knee motion, a patient weight, a patient condition, a knee size, a knee shape, combinations thereof, or the like. The link of the central mechanism can be substantially T-shaped. A linear extension of the link can translate relative to the second support mechanism during the gait cycle.

The first support mechanism can provide support to quadriceps muscles of the patient during a loading response phase of the gait cycle. The first support mechanism can include, e.g., a piston damper, a rotary damper, a passive damper, an elastic material, combinations thereof, or the like. The piston damper can include a dampening structure therein. The dampening structure can include, e.g., a magnetorheological fluid, a spring, combinations thereof, or the like. In some embodiments, the piston damper can include an electromagnet. Varying power supplied to the electromagnet can vary a resistive force imparted by the piston damper.

The second support mechanism can provide support during a stance phase of the gait cycle. In some embodiments, the second support mechanism can provide no support during a free swing phase of the gait cycle. The second support mechanism can include a calf connection. The calf connection can define a planar surface configured to be positioned against a calf of the patient. The calf connection includes a groove track, e.g., a linear groove track, configured to slidably receive therein a complementary protrusion, e.g., a bearing, of the link of the central mechanism. The groove track and the protrusion can form a sliding joint between the central mechanism and the second support mechanism. In some embodiments, the groove track can be substantially linear. In some embodiments, the groove track can be curved.

The link of the central mechanism can include a coupler extending therefrom for interaction with the resistive element. At least a portion of the coupler can translate in and out of the resistive element as the link of the central mechanism translates relative to the second support mechanism during the gait cycle. In some embodiments, the coupler includes a piston extending therefrom. The piston can be configured to be received by an aperture of the resistive element to align translation of the link of the central mechanism relative to the resistive element. The piston can thereby acts as an alignment mechanism between the central mechanism and the second support mechanism. The resistive element can include a resistive device, e.g., a linear spring, a non-linear spring, a magnetic spring, an elastic substrate, combinations thereof, or the like, disposed within a housing.

In accordance with embodiments of the present disclosure, exemplary methods of providing support to a patient during a gait cycle are provided that include providing a knee orthosis device as described above. The methods include providing support during a loading response phase of the gait cycle with the first support mechanism. The methods include providing support during the stance phase of the gait cycle with the second support mechanism by translating at least a portion of the link into the resistive element of the second support element.

In accordance with embodiments of the present disclosure, exemplary knee orthosis devices for providing support to a patient during a gait cycle are provided. The knee orthosis devices include a first support mechanism and a second support mechanism. The second support mechanism includes a calf connection. The calf connection includes a groove track formed therein. The knee orthosis devices include a central mechanism mechanically connecting the first and second support mechanisms. The central mechanism includes a link translatable relative to the second support mechanism during the gait cycle. A complementary protrusion of the link of the central mechanism can slidably translate within the groove track of the second support mechanism during the gait cycle to provide alignment between the central mechanism and the second support mechanism.

In accordance with embodiments of the present disclosure, exemplary knee orthosis devices are provided that generally include a first support mechanism and a second support mechanism for providing support to a patient during a gait cycle. The knee orthosis devices generally include a central mechanism mechanically connecting the first support mechanism and the second support mechanism. The first support mechanism, the second support mechanism and the central mechanism can provide quasi-passive robotic support to a knee of the patient.

The first support mechanism provides support during a loading response phase of the gait cycle. In some embodiments, the first support mechanism provides support to quadriceps muscles of the patient during the loading response phase of the gait cycle. The first support mechanism can include at least one of, e.g., a piston damper, a rotary damper, a passive damper, an elastic material, and the like. The piston damper can include a dampening structure therein. The dampening structure includes at least one of, e.g., a magnetorheological fluid, a spring, and the like. The piston damper generally includes an electromagnet. Varying a power supplied to the electromagnet can vary a resistive force imparted by the piston damper.

The second support mechanism provides support during stance phase of the gait cycle. In some embodiments, the second support mechanism provides support to a knee of the patient during the stance phase of the gait cycle. The second support mechanism generally includes a sliding joint and a resistive element. The resistive element can include a coupler, a housing and a resistive device disposed within the housing. The resistive device can include at least one of, e.g., a linear spring, a non-linear spring, a magnetic spring, an elastic substrate, and the like. In some embodiments, the coupler can be configured and dimensioned to interact with the resistive device to align movement of the coupler relative to the housing.

A level of support provided by at least one of the first support mechanism and the second support mechanism can be adjustable based on at least one of, e.g., a patient knee motion, a patient weight, a patient condition, a knee size, a knee shape, and the like. The central mechanism can be a four-bar linkage system including four links rotatably connected relative to each other. Motion of the four links of the central mechanism can be adjustable based on at least one of, e.g., a patient knee motion, a patient weight, a patient condition, a knee size, a knee shape, and the like.

In accordance with embodiments of the present disclosure, exemplary methods of reducing loads in a knee joint of a patient are provided that generally include providing a knee orthosis device. The knee orthosis device generally includes a first support mechanism and a second support mechanism for providing support to the patient during a gait cycle. The knee orthosis device further includes a central mechanism mechanically connecting the first support mechanism and the second support mechanism. The methods generally include providing support during a loading response phase of the gait cycle with the first support mechanism and providing support during the stance phase of the gait cycle with the second support mechanism. The first support mechanism can include at least one of, e.g., a piston damper, a rotary damper, a passive damper, an elastic material, and the like. The methods can include varying a power supplied to an electromagnet of the piston damper to vary a resistive force imparted by the piston damper.

Other objects and features will become apparent from the following detailed description considered in conjunction with the accompanying drawings. It is to be understood, however, that the drawings are designed as an illustration only and not as a definition of the limits of the invention.

### BRIEF DESCRIPTION OF THE DRAWINGS

To assist those of skill in the art in making and using the disclosed devices and associated methods, reference is made to the accompanying figures, wherein:

FIG. 1 is a side view of an exemplary knee orthosis device according to the present disclosure;

FIG. 2 is a detailed side view of an exemplary knee orthosis device according to the present disclosure;

FIG. 3 is a detailed, wireframe side view of an exemplary knee orthosis device according to the present disclosure;

FIG. 4 is a side view of an exemplary knee orthosis device according to the present disclosure;

FIG. 5 is a detailed side view of an exemplary knee orthosis device according to the present disclosure;

FIG. 6 is a side view of an exemplary knee orthosis device including a compressed spring according to the present disclosure;

FIG. 7 is a detailed side view of an exemplary knee orthosis device including a compressed spring according to the present disclosure;

FIG. 8 is a side view of an exemplary knee orthosis device including an expanded spring according to the present disclosure;

FIG. 9 is a detailed side view of an exemplary knee orthosis device including an expanded spring according to the present disclosure;

FIG. 10 is a side view of an exemplary knee orthosis device including a damper according to the present disclosure;

FIG. 11 is a side view of an exemplary knee orthosis device including a damper according to the present disclosure;

FIG. 12 is a chart illustrating reduction of knee moment due to actuation of a spring and damper of an exemplary knee orthosis device according to the present disclosure; and

FIG. 13 is a chart illustrating reduction of contact force due to a spring of an exemplary knee orthosis device according to the present disclosure.

### DESCRIPTION OF EXEMPLARY EMBODIMENTS

As discussed above, at points during the loading response and the midstance phase of the gait cycle, the knee can encounter loads higher than the total body weight, as well as significant demands on the quadriceps extensor muscles. Reduction of these loads and moments generally lowers pain experienced in the knees of those suffering from osteoarthritis and may provide a favorable environment for joint repair. Accordingly, muscle support can be critical during loading response and knee support can be critical during the entire stance phase.

The quasi-passive robotic orthosis device of the present disclosure, when worn by a patient, generally reduces the forces transferred through the knee during everyday activities and provides a supporting moment to the muscles of the thigh. Thus, the exemplary orthosis device provides knee support during the entire stance phase and muscle support during loading response. The knee and muscle support provided by the exemplary orthosis device can advantageously be used in a variety of application for support and stabilization. For example, the orthosis device can be used to relieve or reduce pain from osteoarthritis. Additional applications include ailments that affect mobility, such as stroke, other forms of arthritis, muscle weakness, ailments that necessitate muscle control, slight nerve damage, beginning of multiple sclerosis (MS), and alternative similar ailments. Further applications include rehabilitation or physical therapy from surgery and/or injury to stabilize and reduce joint pain, and sport use, such as extreme sports (e.g., motocross, skiing, and the like), baseball catchers, golf, and other similar sports, where an athlete risks potential injury or has mild knee pain. Additional applications include prophylactic uses, such as high stress jobs (e.g., construction, agriculture, military, industrial, and the like) where a worker risks potential injury or requires additional support of the knee and/or muscles for performing the job. Those of ordinary skill in the art should understand that the exemplary orthosis device discussed herein can be used for a variety of alternative applications.

In some embodiments, the quasi-passive robotic orthosis device does not include actuators, e.g., motors. Instead, the orthosis device provides an ability to automatically modulate the stiffness of the support elements in such a way as to provide support during key phases of the gait cycle, while allowing free range of motion in other phases of the gain cycle. As will be discussed in greater detail below, this modularity can be accomplished by combining design elements, such as a knee tracking mechanism, a passive load acceptance system, and a resistive element, in the device.

The knee tracking mechanism generally allows the device to follow the natural motion of the patient's knee. The passive load acceptance system generally absorbs a portion of the force experienced by the knee joint. The resistive element generally provides a supporting moment to the quadriceps muscles. The exemplary orthosis device can be customized to a patient based on, e.g., the patient's size, shape, motion, and condition, to relieve pain caused by osteoarthritis and other ailments, as well as to provide support to increase stability. For example, the orthosis device can be customized to a patient based on the unique knee motion of the patient. This customization may be accomplished by analyzing motion data from the patient and adjusting the orthosis device to follow the precise motions of the patient, thereby increasing comfort. In some embodiments, the specific condition for which the patient is being treated can be taken into account when customizing the orthosis device by incorporating slight changes to the resistive elements and/or the control system such that the orthosis device provides the requisite support to the patient.

The exemplary orthosis device can be based on a quasi-passive system. In particular, while using the orthosis device, the patient can move under their own power and the orthosis device can react and support those movements. Thus, the orthosis device does not dictate movement. Rather, in contrast to the motorized solutions discussed above in which movement is created by the device, e.g., powered leg exoskeletons, powered wheelchairs, and the like, the exemplary orthosis device assists in the movement created and dictated by the patient. The avoidance of actuators generally allows for low power consumption by the orthosis device and an overall lighter, easier to use and easier to control device. In some embodiments, rather than implementing actuators, a passive load assistance system can be implemented to provide support and act as a shock absorber for the knee.

The robotic orthosis device can be worn over the knee in a manner similar to most athletic braces. The technical description of the orthosis device can be broken down into two sections: state recognition and mechanical design.

A state recognition system can provide the orthosis device with enough information to reliably activate at the right time. An exemplary strategy can be to track the phase-of-gait of a particular patient, as well as the angular position and velocity of the knee. The measurements which are generally needed for the phase-of-gait, the angular position of the knee and the velocity of the knee are the knee flexion angle, the knee angular velocity, and the pressure distribution of the foot. An exemplary method of measuring pressure distribution can include using eight foot contact sensors, four on each foot. (See, e.g., Kong, K. et al., A Gait Monitoring System Based on Air Pressure Sensors Embedded in a Shoe, IEEE/ASME Transactions on Mechatronics, Vol. 14, No. 3, pp. 358-370 (June 2009)). It should be understood that in some embodiments, less or more contact sensors can be used on each foot to measure the pressure distribution. Using the pressure distribution information from these sensors, the phase-of-gait of a patient can be determined.

The orthosis device should also be able to recognize a more detailed state within a certain phase. This can be performed by measuring the knee angle and the knee angular velocity with, e.g., a position sensor. In some embodiments, the orthosis device can react or adjust the support provided based on received or detected angle sensor data, displacement sensor data, or both, interpreted through a microprocessor powered by an energy source, such as a battery. In some embodiments, one or more sensors can be positioned on the orthosis device to allow the orthosis device to automatically adjust the support provided to the user based on the received sensor data. Determining the phase-of-gait, the knee angle, and the knee angular velocity generally provides sufficient information to intelligently activate the orthosis device in a customized manner for the particular patient. One benefit of the above-described technique may be that the measurements taken allow activities other than basic walking to be recognized and classified. Thus, for example, based on the measurements taken, the orthosis device can be customized to provide the requisite support when a patient moves up and down the stairs.

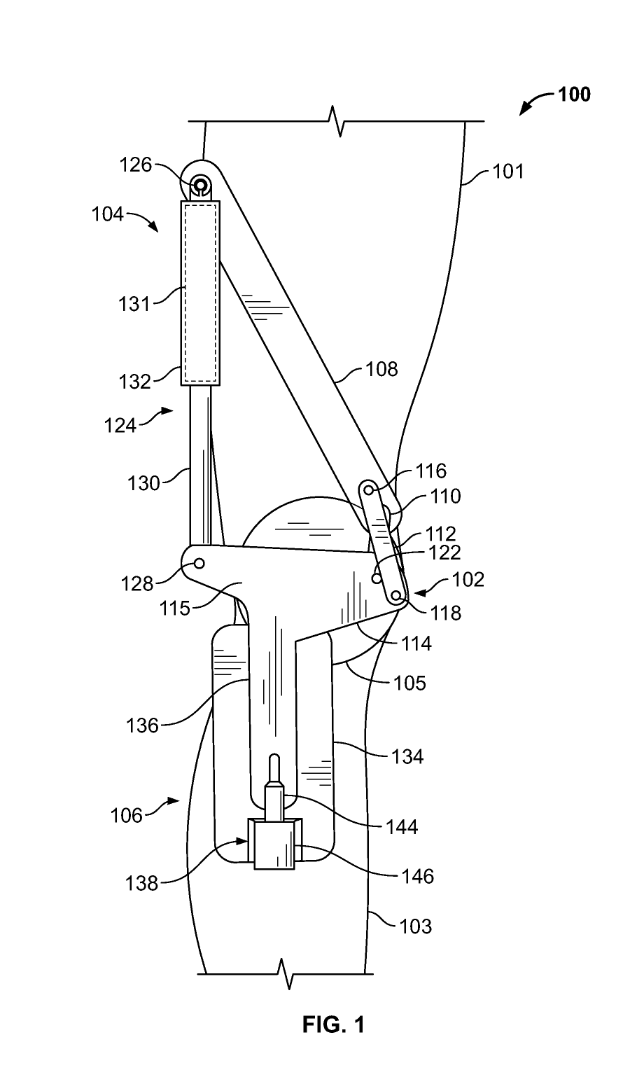

Turning now to FIG. 1, a side view of an exemplary knee orthosis device 100 or brace is provided. The mechanical design of the orthosis device 100 can incorporate one or more of the design components discussed below. An exemplary design component can be a central mechanism 102 which connects the upper leg portion mechanism 104 to the lower leg portion mechanism 106 of the orthosis device 100. The upper leg portion mechanism 104 can connect or strap on to the upper leg portion 101, e.g., the thigh, and the lower leg portion mechanism 106 can connect or strap on to the lower leg portion 103, e.g., the calf, to provide the necessary support for the knee 105. The upper leg portion mechanism 104 can provide muscle support to the upper leg portion 101 and the lower leg portion mechanism 106 can provide an unloading mechanism to the user.

The central mechanism 102 can faithfully follow the knee's 105 natural motions during gait, thereby acting as a knee tracking mechanism. This can be accomplished with one or more mechanisms working either together or in parallel. In some embodiments, the central mechanism 102 can be a multi-bar linkage or cam system. For example, the central mechanism 102 of FIG. 1 is illustrated as a four-bar linkage or cam system, e.g., a first link 108, a second link 110, a third link 112, and a fourth link 114. In some embodiments, the first link 108 can be referred to as the thigh link and the second, third and fourth links 110, 112, 114 can be referred to as coupler links. The first, second, third and fourth links 108, 110, 112, 114 can be hingedly and rotationally connected relative to each other to permit knee 105 tracking motion during the gait cycle.

The first link 108 can define an elongated link extending from the knee 105 joint to the thigh of the patient. The second and third links 110, 112 can define elongated links and hingedly connect the fourth link 114 to the first link 108. In the embodiment of FIG. 1, the length of the first link 108 can be greater than the length of the second and third links 110, 112. The length of the third link 112 can be greater than the length of the second link 110. The fourth link 114 can define a substantially T-shaped configuration, including a horizontal portion 115 extending along the knee 105 joint and an extension 136, e.g., a vertical extension, extending from the knee 105 joint along the calf of the patient. The horizontal portion 115 of the fourth link 114 can include angled surfaces on either size of the extension 136 which connect with the extension 136. For example, the angled surface on the left of the extension 136 can connect at a location closer to the knee 105 joint along the extension 136 than the angled surface on the right of the extension 136. Thus, the width of the horizontal portion 115 can be greater on the right side of the extension 136 as compared to the left side of the extension 136 to permit appropriate connection of the second and third links 110, 112. However, it should be understood that alternative configurations of the fourth link 114 can be used based on the desired motion of the components of the central mechanism 102, as long as the fourth link 114 includes the extension 136 for engaging a resistive element 138 of the lower leg portion mechanism 106.

The four-bar linkage central mechanism 102 can be designed with small modifications to be customized to any patient's knee 105 motion. For example, a position of one or more of the joints 116, 118 of the third link 112 may be modified relative to the first link 108 and the fourth link 114 such that the overall motion of the four-bar linkage central mechanism 102 can be varied to accommodate a patient's knee 105 motion. It should be understood that one or more joints of the remaining links can also be adjusted depending on the knee 105 motion to be replicated. In some embodiments, the length of the first, second, third and/or fourth links 108, 110, 112, 114 may be varied to customize the motion of the components of the central mechanism 102. The four-bar linkage central mechanism 102 can thereby complement the other components of the orthosis device 100, while allowing normal motion during gait.

The four-bar linkage central mechanism 102 can also effectively transfer force from the lower leg portion mechanism 106 to the upper leg portion mechanism 104 of the orthosis device 100. In particular, during the gait cycle, rotation or movement of the components of the central mechanism 102 can adjust whether forces are imparted on and/or support is provided by the lower leg portion mechanism 106, the upper leg portion mechanism 104, or both. The appropriate support mechanism can thereby be used during different phases of the gait cycle to provide effective support to the patient. Thus, the exemplary four-bar linkage central mechanism 102 provides a customization aspect of the orthosis device 100 and provides an optimal force transfer between the components of the orthosis device 100.

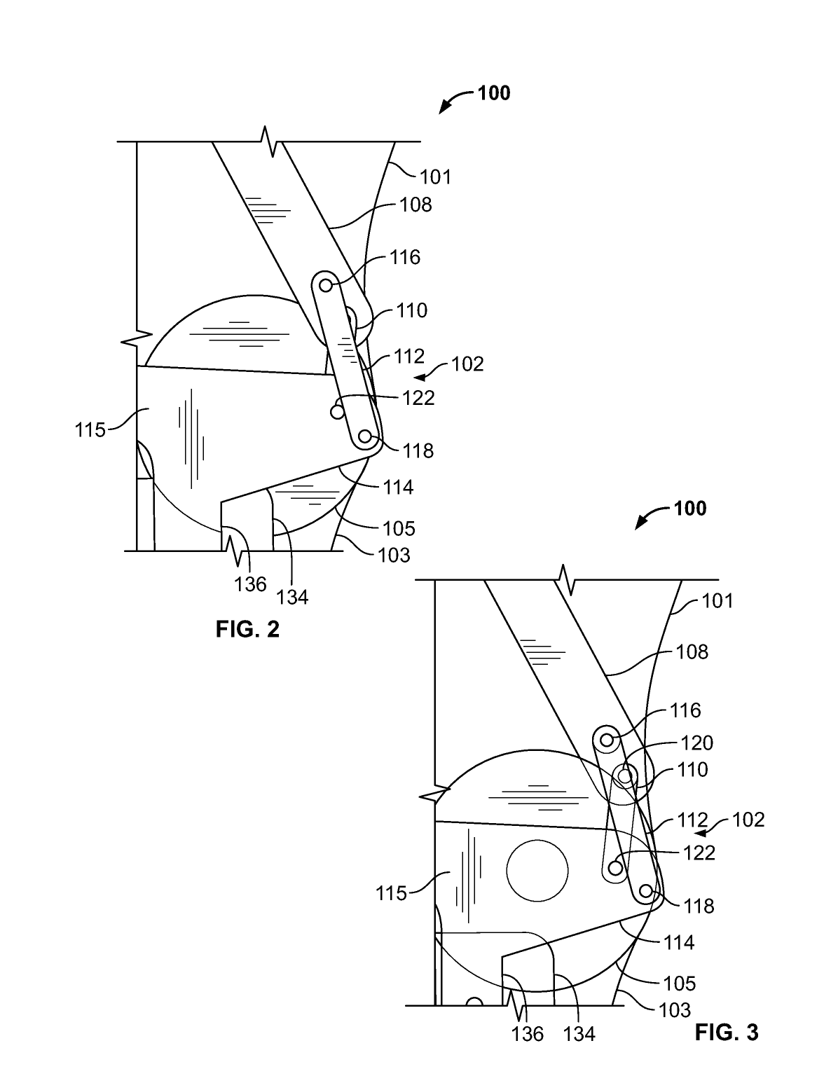

FIG. 2 shows a detailed side view of the orthosis device 100 and FIG. 3 shows a detailed, wireframe side view of the orthosis device 100. As can be seen from FIG. 3, the second link 110 can rotatably connect to the first link 108 at joint 120 and to the fourth link at joint 122. Similarly, the third link 112 can rotatably connect to the first link 108 at joint 116 and to the fourth link at joint 118. As the patient moves through the gait cycle, each of the linkages can hingedly rotate at the respective joint to accurately track the motion of the knee 105. By accurately tracking the motion of the knee 105, the upper and lower leg portion mechanisms 104, 106 can provide the necessary support to the knee 105 and the associated muscles at each phase of the gait cycle.

Still with reference to FIG. 1, the four-bar linkage central mechanism 102 can connect the upper leg portion 101 or thigh support mechanism, e.g., the upper leg portion mechanism 104, and the lower leg portion 103 support mechanism or calf support mechanism, e.g., the lower leg portion mechanism 106. The upper leg portion mechanism 104 can be a first support mechanism and the lower leg portion mechanism 106 can be a second support mechanism of the orthosis device 100. The upper leg portion mechanism 104 can be detachably connected or strapped on to the upper leg portion 101 of the patient with, e.g., VELCRO® straps, and the like. In some embodiments, the upper leg portion mechanism 104 can be a resistive element and the lower leg portion mechanism 106 can be a passive load acceptance system of the orthosis device 100.

The upper leg portion mechanism 104 can include a piston damper 124 or shock absorber, e.g., a magnetorheological (MR) damper, a spring-loaded sliding support, and the like, connected to the first link 108 and the fourth link 114 at joints 126 and 128, respectively. In some embodiments, a thigh connection or attachment component can be attached to the upper leg portion mechanism 104 by the piston damper 104, the first link, or both. As will be described in greater detail below, the piston damper 124 can include a dampening structure 131, e.g., a spring, MR fluid, and the like, therein which regulates the sliding interaction between a first rod 130 and a second rod 132. During the different phases of the gait cycle, the sliding interaction between the first and second rods 130, 132 can be along a vertical axis in a substantially parallel direction relative to the upper leg portion 101 of the patient. For example, the first rod 130 can slide in and out of the second rod 132 in a substantially vertical direction parallel the upper leg portion 101.

The lower leg portion mechanism 106 of the orthosis device 100 generally includes a calf connection 134 which can be detachably connected or strapped on to the lower leg portion 103 of the patient with, e.g., VELCRO® straps, and the like. The calf connection 134 can define a substantially rectangular shape and, in some embodiments, can include rounded corners. In some embodiments, the width of the calf connection 134 can be dimensioned large enough to provide a supporting surface against the lower leg portion 103 of the patient. The calf connection 134 can thereby be positioned or strapped to the lower leg portion 103 in a manner which reduces or prevents movement of the calf connection 134 relative to the lower leg portion 103. In some embodiments, rather than a planar calf connection 134, the calf connection 134 can define a curved or deformable shape to permit the configuration of the calf connection 134 to be adjusted based on the curvature of the lower leg portion 103. In some embodiments, a middle portion of the calf connection 134 which interacts with the central mechanism 102 can be substantially stiff and side portions of the calf connection 134 can be fabricated from a deformable material which can be adjusted to conform to the lower leg portion 103 of the patient.

As will be discussed in greater detail below, the calf connection 134 can act as a load bearing system which takes up at least a portion of the load felt by the knee 105 during the stance phase. The calf connection 134 can include a groove track therein for mechanical interaction with the extension 136 of the fourth link 114. In particular, a bearing or protrusion at one end of the fourth link 114 can be complementary to the groove track of the calf connection 134 such that during the gait cycle the fourth link 114 can slide within the groove track to form a sliding joint as the central mechanism 102 components move. In some embodiments, the lower leg portion mechanism 106 can include a resistive element 138 which provides additional or alternative support to the knee 105 of the patient during the gait cycle.

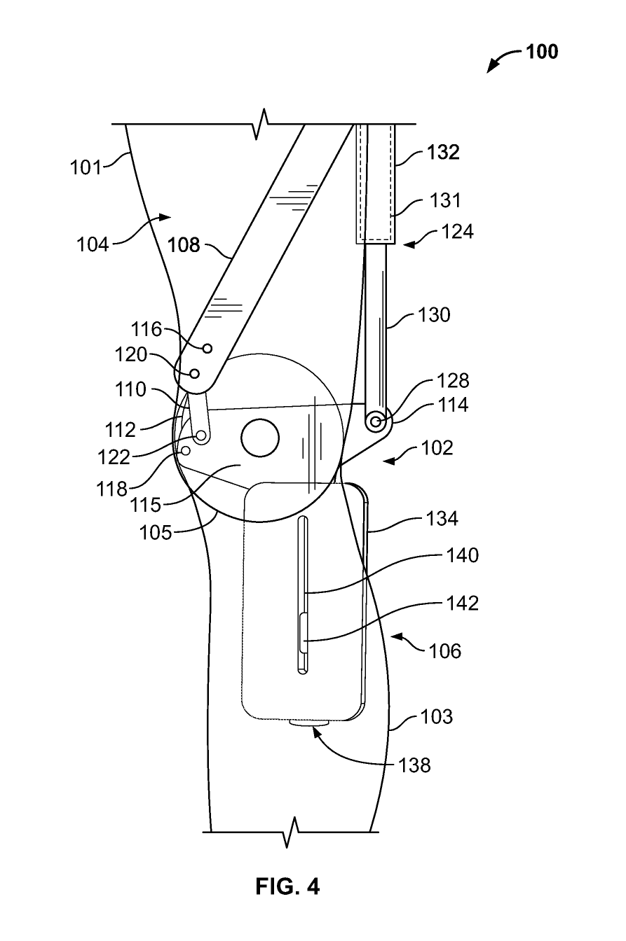

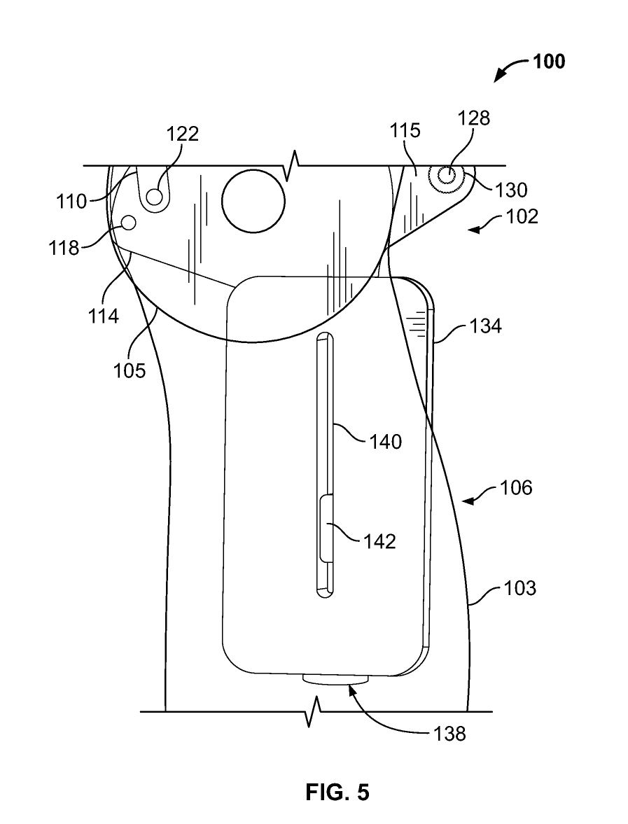

Turning now to FIGS. 4 and 5, side views of the exemplary orthosis device 100 are provided. In particular, FIGS. 4 and 5 illustrate the orthosis device 100 from a side opposing that shown in FIGS. 1-3. Thus, the opposing view of the lower leg portion mechanism 106 positioned against the lower leg portion 103 of the patient can be seen. As discussed above, the lower leg portion mechanism 106 generally includes a sliding joint or link formed by the mechanical interaction of the fourth link 114 and the calf connection 134. As shown in FIGS. 4 and 5, the calf connection 134 includes a groove track 140 formed therein. The groove track 140 can be substantially linear and can extend vertically relative to the height of the calf connection 134. For example, the groove track 140 can extend along a vertical axis in a direction substantially parallel to the length of the lower leg portion 103. The groove track 140 can be configured and dimensioned to slidably receive therein a protrusion 142, e.g., a bearing, of the fourth link 114 extension 136. The extension 136 of the fourth link 114 can therefore translate in a substantially linear manner. However, it should be understood that alternative non-linear groove tracks 140 can be implemented, resulting in non-linear translation of the extension 136 of the fourth link 114. In some embodiments, the protrusion 142 can be releasably fitted into the groove track 140 and can slide along the groove track 140 as the lower leg portion 103 of the user moves, thereby facilitating motion of the components of the central mechanism 102.

FIGS. 4 and 5 illustrate the groove track 140 as a uniform depth cut-out from the calf connection 134. In some embodiments, the groove track 140 can define a substantially T-shaped configuration extending a partial distance through the thickness of the calf connection 134. In such embodiments, the protrusion 142 of the extension 136 can define a complementary T-shaped form and can be inserted into the groove track 140 for slidable interaction with the calf connection 134. In some embodiments, the calf connection 134 can be provided with a protrusion 142 within the groove track 140 and the protrusion 142 can be connected to the extension 136 prior to use of the orthosis device 100.

In some embodiments, the dimensions of the protrusion 142 can be changed to vary the degree of motion permitted by the sliding joint of the calf connection 134. For example, the protrusion 142 can be decreased in length to permit the fourth link 114 extension 136 to slide a greater distance relative to the calf connection 142. Similarly, the protrusion 142 can be increased in length to reduce the distance the fourth link 114 extension 136 can slide relative to the calf connection 134. In some embodiments, the calf connection 134 can be interchangeable to vary the degree of motion permitted by the sliding joint. For example, the interchangeable calf connections 134 can define different lengths of the groove track 140. The sliding joint interaction between the calf connection 134 and the fourth link 114 can thereby be customized based on the gait cycle of the patient.

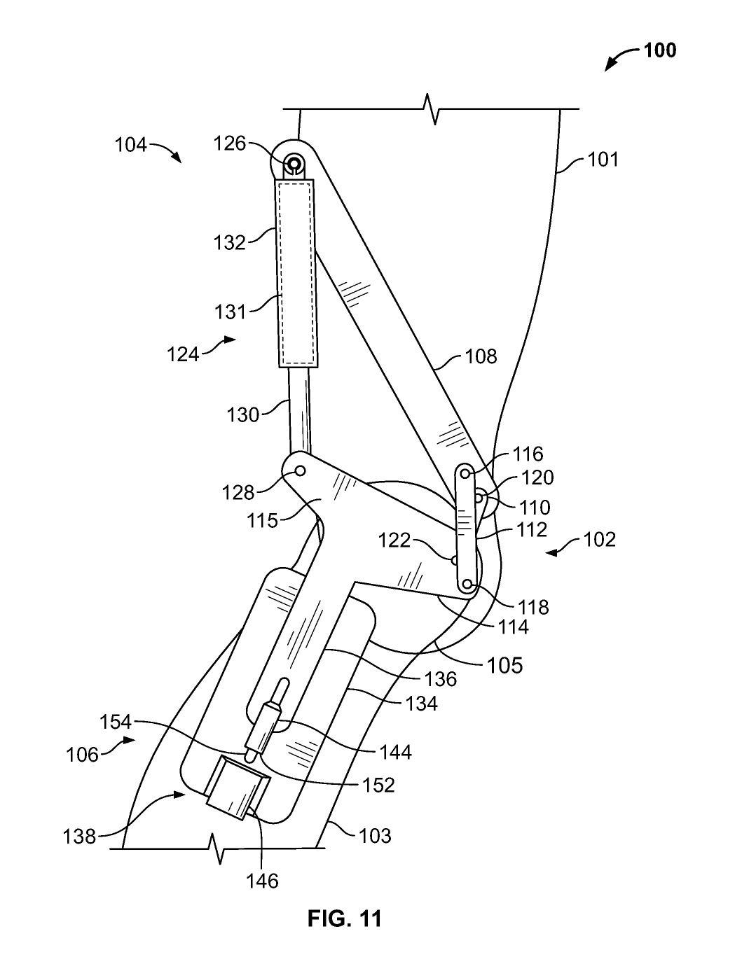

During the stance phase of the gait cycle, the central mechanism 102 can extend the fourth link 114 extension 136 to the lowermost or near lowermost position along the groove track 140, thereby positioning the protrusion 142 furthest from the knee 105 joint. (See, e.g., FIG. 4). Alternatively, during the free swing phase or bent position of the knee, the central mechanism 102 can track the motion of the knee 105 and position the fourth link 114 extension 136 to the uppermost or near uppermost position along the groove track 140, thereby positioning the protrusion 142 closest to the knee 105 joint. (See, e.g., FIG. 11). Based on the gait cycle of the patient, a calf connection 134 with the appropriate groove track 140 length can be selected. Thus, the variable connection by the sliding joint of the fourth link 114 of the central mechanism 102 and the calf connection 134 creates a mechanical system capable of following the motion of the knee 105 of a patient.

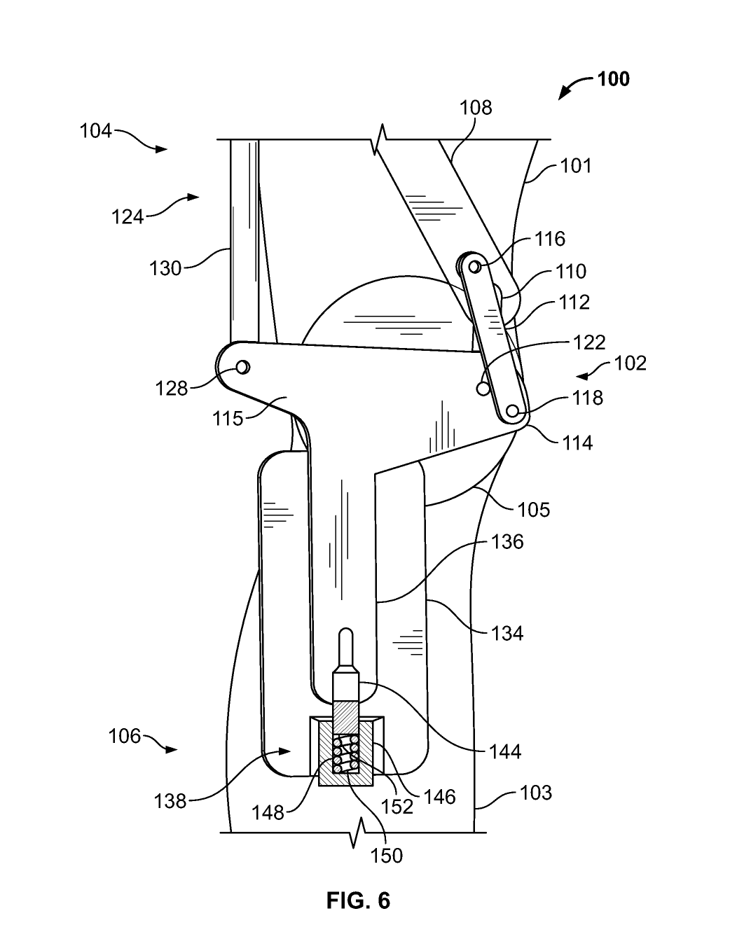

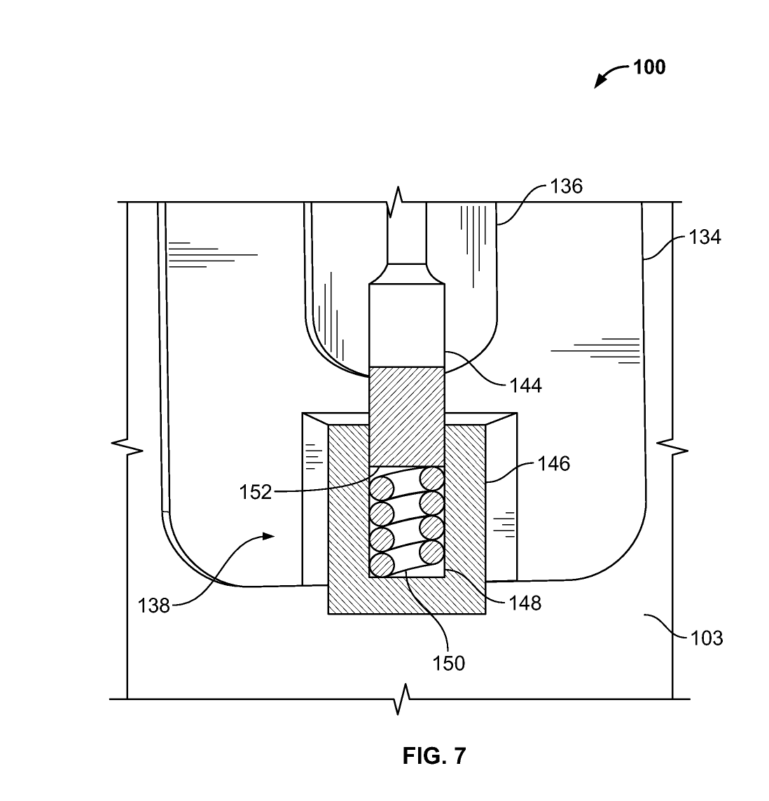

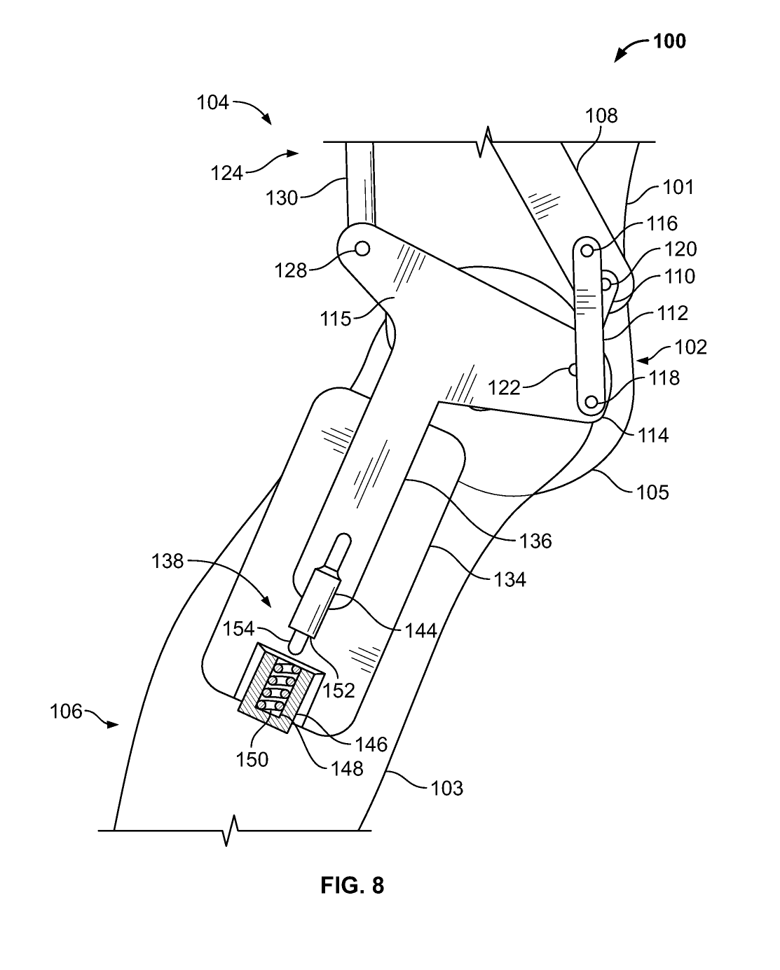

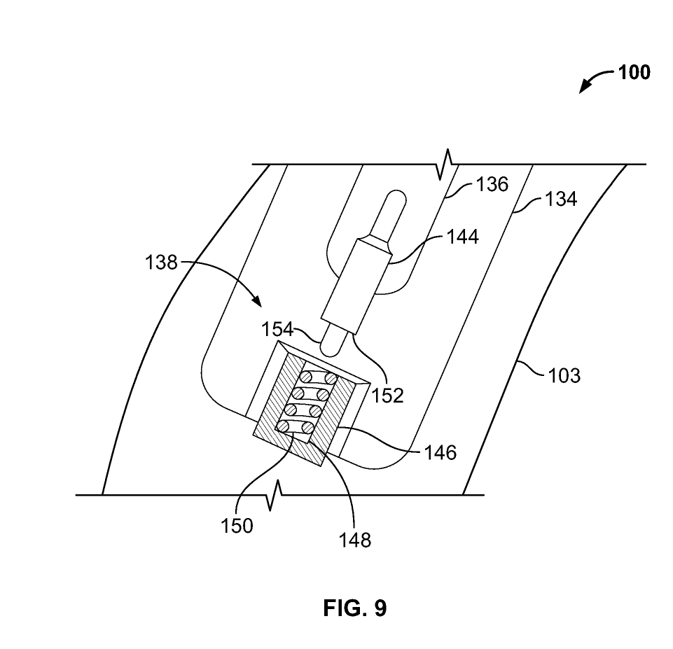

With reference to FIGS. 6-9, side and detailed views of the exemplary orthosis device 100 are provided. In particular, FIGS. 6-9 illustrate a side view of the orthosis device 100 with a cross-sectional view of the resistive element 138, e.g., a load bearing system for the lower leg portion 103. As the load bearing system, the resistive element 138 can take up at least a portion of the load felt by the knee 105 during the stance phase (FIGS. 6 and 7), while allowing substantially free swing during the gait (FIGS. 8 and 9). This can be achieved by placing a resisting structure, e.g., a damping structure, between the fourth link 114 of the central mechanism 102 and the leg or calf connection 134. Thus, the resistive element 138 can mechanically interact with the sliding joint formed by the extension 136 and the calf connection 134 described above to provide the requisite support to the knee 105.

In some embodiments, the resistive element 138 can include a coupler 144 connected to and extending from a distal end of the fourth link 114 extension 136. The coupler 144 can be aligned with the vertical axis of the extension 136 such that a portion of the coupler 144 extends beyond the distal end of the extension 136. The resistive element 138 can include a housing 146 connected to and extending from the calf connection 134. The resistive element housing 146 generally includes a cavity 148 therein configured and dimensioned to receive a damping or resistive device 150, e.g., a linear spring, a non-linear spring, a magnetic spring, an elastic substrate, combinations thereof, and the like.

The distal end of the coupler 144 extending beyond the distal end of the extension 136 can be configured and dimensioned to translate in and out of the cavity 148 to impart a force on the resistive device 150. In particular, the coupler 144 can translate in and out of the cavity 148 as the extension 136 slides along the groove track 140 of the calf connection 134 due to the knee 105 motion tracking of the central mechanism 102. In some embodiments, the distal end of the coupler 144 and the cavity 148 of the housing 146 can define complementary cylindrical forms which permit the cavity 148 to at least partially receive the distal end of the coupler 144 therein. For example, as shown in FIG. 6, during the stance phase of the gait cycle, the distal end of the coupler 144 can be translated into the cavity 148 and imparts a force against the resistive device 150 based on the amount of translation of the extension 136 along the groove track 140. The resistive device 150 can, in turn, impart a force on the distal end of the coupler 144 to provide support for the knee 105.

As would be understood by those of ordinary skill in the art, the resistive device 150 can be interchangeable with alternative resistive devices 150 which provide varying degrees of support and/or weight reduction. In embodiments implementing a spring as the resistive device 150, a spring with a particular spring constant can be selected to provide the desired support to the knee 105. For example, if a greater amount of support is desired, a spring with a higher spring constant can be selected to resist the force of the coupler 144. Similarly, if a smaller amount of support is desired, a spring with a lower spring constant can be selected to provide less resistance to the force of the coupler 144. Thus, the orthosis device 100 can be customized for the particular motion, support and/or weight reduction required for the patient.

In some embodiments, the resistive device 150 can be a spring, e.g., a non-linear spring fabricated from titanium. In some embodiments, the spring can be manufactured by a three dimensional printer. Three dimensional printing of a spring can allow for advantageous customization of the forces required to support the knee 105. In some embodiments, the resistive device 150, e.g., the spring, can be passive and only activated when the foot of the patient is on or about to be in contact with the ground. In particular, the spring can be configured and dimensioned to match the necessary forces during the gait cycle of the patient and can provide a constant weight reduction percentage during the stance phase. The passive support of the resistive element 138 can be ensured by the relative motion between the coupler 144 and the housing 146 of the calf connection 134, as well as the selection of a proper neutral spring length for use as the resistive device 150. In particular, the amount of translation permitted by the extension 136 along the groove track 140 of the calf connection 134 can be adjusted to vary the amount of relative motion between the coupler 144 and the housing 146. In some embodiments, the position of the housing 146 on the calf connection 134 along the translation path of the coupler 144 can be adjusted by the user to vary the relative motion between the coupler 144 and the housing 146.

Thus, when the leg is substantially extended during the stance phase of the patient as the foot of the patient makes contact with the ground during walking (FIGS. 6 and 7), the spring can be activated to undergo a maximum compression by the coupler 144 and can, in turn, provide support to the knee 105. For example, FIGS. 6 and 7 illustrate the maximum compression of the spring at approximately 0° of flexion during the gait cycle, thereby providing the maximum amount of support to the knee 105. It should be understood that as the gait cycle changes, the resistive device 150 can provide a variable amount of support to the knee 105 up to the maximum support provided at the position of maximum compression of the resistive device 150.

When the leg is bent at the knee 105 when the foot of the patient is in the air during a swing of the leg (FIGS. 8 and 9), the coupler 144 can translate substantially out of the housing 146 to permit a maximum expansion or deactivation of the spring. For example, when the leg is bent at the knee 105, contact between the coupler 144 and the spring can be prevented. The spring can thereby provide the least amount of or no support during the gait of the patient to allow for a substantially free swing of the leg. In some embodiments, the housing 146 can be dimensioned to house the distal end of the coupler 144 even during a bend in the knee 105. For example, the distal end of the coupler 144 can translate away from the resistive device 150 during bending of the knee 105 to a point where the resistive device 150 reaches a maximum expansion or provides no support to the knee 105, and the distal end of the coupler 144 can remain within the side walls of the housing 146. Alignment between the coupler 144 and the housing 146 can thereby be maintained. As noted above, it should be understood that between the substantially extended position of FIGS. 6 and 7 and the bent position of FIGS. 8 and 9, the resistive device 150 can provide a varied amount of support as the leg moves during the gait cycle.

In some embodiments, a force-imparting surface 152, e.g., the distal end or near-distal end, of the coupler 144 can be configured as substantially flat to provide a uniform surface which interacts with a top surface of the resistive device 150 (see, e.g., FIGS. 6 and 7). In some embodiments, the force-imparting surface 152 of the coupler 144 can include a piston 154 extending therefrom (see, e.g., FIGS. 8 and 9) for interaction with the resistive device 150. For example, in embodiments implementing a spring as the resistive device 150, the piston 154 can act as an alignment element or mechanism by entering the aperture formed by the spring, thereby ensuring that the coupler 144 enters the cavity 148 of the housing 146 in an aligned manner. In particular, as the coupler 144 translates with the extension 136 in the direction of the resistive device 150, the piston 154 can enter the aperture formed by the coils of the spring to ensure that the force-imparting surface 152 enters the cavity 148 and uniformly applies a force against the resistive device 150. In embodiments implementing an alternative resistive device 150, the resistive device 150 can include an aperture complementary to the piston 154 to ensure the proper alignment of the coupler 144 with the housing 146.

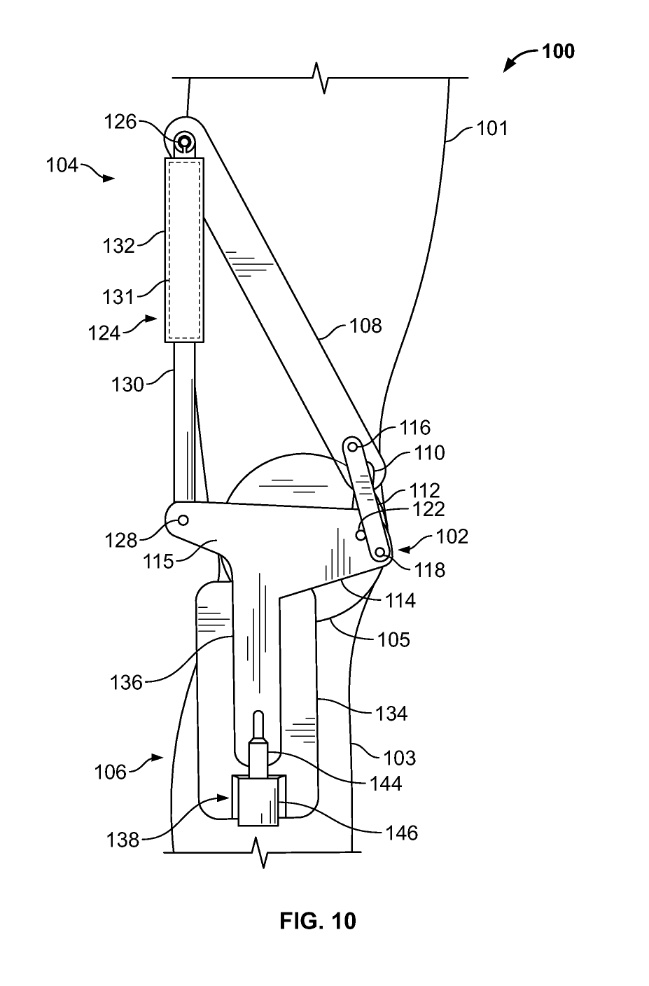

With reference to FIGS. 10 and 11, side views of the exemplary orthosis device 100 are provided. In particular, FIG. 10 illustrates a side view of the orthosis device 100 during the stance phase, while FIG. 11 illustrates a side view of the orthosis device 100 during the free swing of the gait cycle. As discussed above, the orthosis device 100 reinforces the quadriceps muscles of the upper leg portion 101 or thigh during the loading response phase. This reinforcement can take the form of a supportive torque about the knee 105 joint by a resistive element. In some embodiments, a piston damper 124 containing a dampening structure 131 therein, e.g., a variable electromagnet, a spring, a magnetorheological (MR) fluid (e.g., an MR damper), and the like, can provide the requisite torque.

The dampening structure 131 can regulate the sliding interaction between a first rod 130 and a second rod 132 of the piston damper 124. In particular, the first rod 130 can be axially aligned with the second rod 132 and can concentrically slide within an aperture formed in the second rod 132 as the links of the central mechanism 102 move relative to each other during the gait cycle. As illustrated in FIGS. 10 and 11, one or both of the first and second rods 130, 132 can translate along a vertical axis relative to each other substantially parallel to the upper leg portion 101 of the patient. In some embodiments, the dampening structure 131 can be positioned within the second rod 132 and can regulate the resistive force imparted on the first rod 130 as it axially translates within the second rod 132. In some embodiments, the dampening structure 131 can be positioned within the first rod 130. In some embodiments, the dampening structure 131 can be positioned in both the first and second rods 130, 132. The dampening structure 131 can thereby provide a varying supporting force to the knee 105 of the patient during different phases of the gait cycle.

The piston damper 124 can be placed between any two links of the central mechanism 102. In some embodiments, the piston damper 124 can be positioned between two moving links or components, e.g., the first link 108 and the fourth link 114, and the like, to provide effective support. In particular, although illustrated as being positioned above the knee 105 and coupled to the first link 108 and the fourth link 114, it should be understood that the piston damper 124 can be placed between any two links or parts that have relative motion, e.g., two links, above the knee 105 and with the lower leg portion mechanism 106, the central mechanism 102 and the lower leg portion mechanism 106, and the like.

In some embodiments, the piston damper 124 can be placed to connect the thigh and coupler links. For example, as illustrated in FIGS. 10 and 11, the piston damper 124 can connect to the first link 108 at joint 126 and to the fourth link 128 at joint 128. The resistive force imparted by the piston damper 124 can be varied quickly and in a predictable manner to customize the orthosis device 100 for a particular patient. For example, the resistive force imparted by the piston damper 124 can be varied by controlling the power supplied to an electromagnet located in the piston damper 124. Thus, the piston damper 124 can be customized based on a patient's strength and/or condition. When active, the piston damper 124 can be controlled to provide support to the patient. When the piston damper 124 is deactivated or off, the piston damper 124 can provide a minimal amount of resistance, thereby allowing the knee to swing freely. In some embodiments, the piston damper 124 can automatically deactivate and activate based on the sensed phase of the gait cycle of the patient. In some embodiments, the piston damper 124 can be manually deactivated and activated based on the preference of the patient.

In some embodiments, rather than or in combination with the piston damper 124, support can be provided to the muscles of the patient by implementing a MR rotary damper, a passive damper, an elastic material, combinations thereof, and the like. A selection of the appropriate damping mechanism can be made based on, e.g., size, controllability, capacity for producing force, and the like. As an example, as illustrated in FIG. 10, when the patient fully extends the leg, such as during extension associated with a stance position, the first rod 130 can be extended out of the second rod 132 to a maximum distance and the piston damper 124 can impart a maximum supporting force. As illustrated in FIG. 11, when the patient fully bends the leg at the knee 105, the first rod 130 can be retracted into the second rod 132 to a maximum distance and the piston damper 124 can impart a minimum supporting force.

The knee 105 and thigh can therefore receive the necessary support from the piston damper 124 during extension of the leg in the response phase. In some embodiments, the degree and timing of the dampening force imparted by the piston damper 124 can be regulated by the electromagnet (or alternative dampening structure 131) located within the piston damper 124, such that the piston damper 124 can become more or less stiff as desired, or be turned off when not needed. It should be understood that between the fully extended position and the fully bent position of the leg, the piston damper 124 can provide a varying degree of support to the knee 105 and thigh based on, e.g., the compression or configuration of the internal dampening structure 131, the amount of actuation of an electromagnet located in the piston damper 124, and the like.

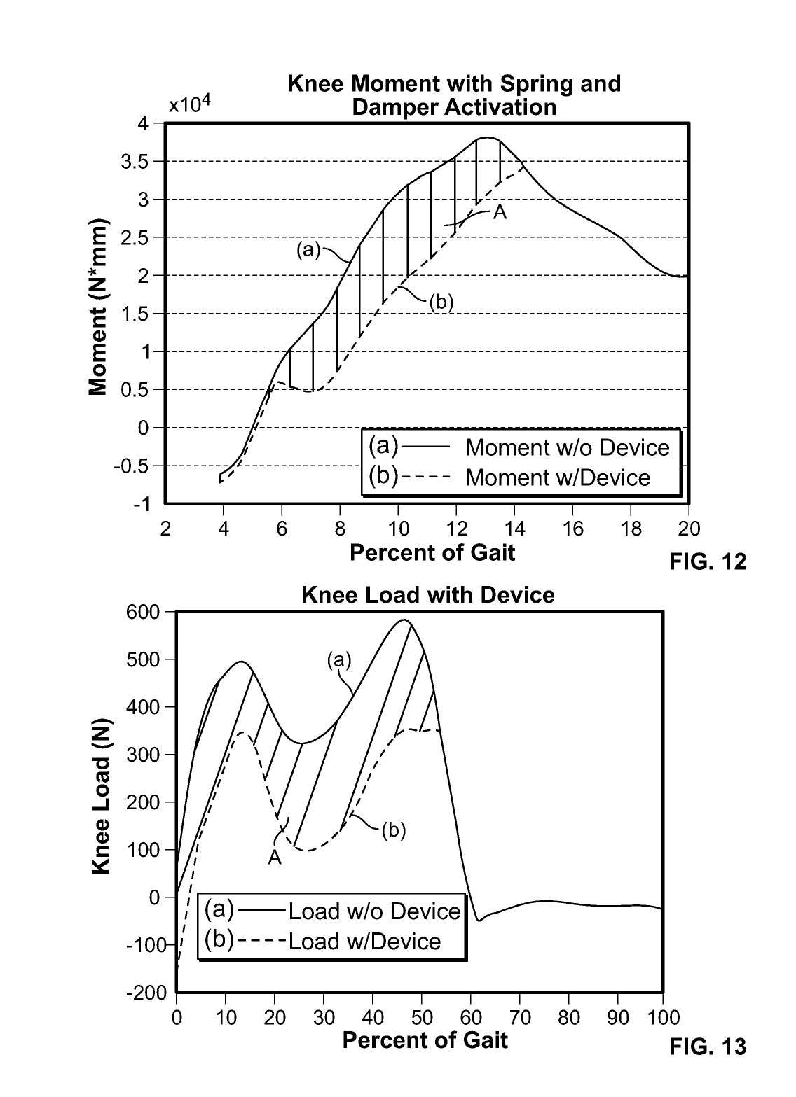

Experimentation of the exemplary orthosis device 100 was performed via a two dimensional kineto-static simulation of a gait cycle. Motion capture and ground reaction force data was processed to show the motion of the leg in the sagittal plane. (See, e.g., Winter, D. A., Biomechanics and Motor Control of Human Movement, Third, Hoboken, N.J., John Wiley & Sons, Inc. (2005); Carnegie Mellon University—CMU Graphics Lab—Motion Capture Library, available at http://mocap.cs.cmu.edu/ (October 2012); and MATLAB, Natick, Mass., The MathWorks, Inc. (2013)). Using the data relating to the motion of the leg and the external forces being exerted on the system, the reaction forces and muscle moments at the knee 105 were calculated. The orthosis device 100 was then added to the simulation and, as illustrated in the charts of FIGS. 12 and 13, reductions in overall joint contact force and muscle moment were observed.

With respect to FIG. 12, a reduction in muscle moment demand in the knee 105 due to activation of the orthosis device 100 is provided. Curve (a) of FIG. 12 shows the moment demand in the knee 105 without implementation of the orthosis device 100, while curve (b) shows the moment demand in the knee 105 with the orthosis device 100. Area A represents the total reduction of the moment demand in the knee 105. As can be seen from FIG. 12, during the critical period between approximately 5% and approximately 20% of the gait cycle, the orthosis device 100 was found to substantially reduce the moment demand in the knee 105. (See, e.g., Pollo, F. E. et al., Reduction of Medial Compartment Loads with Valgus Bracing of the Osteoarthritic Knee, Am. J. Sports Med., Vol. 30, pp. 414-421 (2002)). For example, on average, the orthosis device 100 was found to reduce the moment demand in the knee 105 by approximately 1.1×10⁴N·mm. However, as discussed herein, it should be understood that the support provided by the exemplary orthosis device 100 may be customized based on the needs of a patient and, therefore, the resulting performance of the orthosis device 100 can vary.

With respect to FIG. 13, a reduction in load or contact force experienced by the knee 105 due to activation of the resistive element 138 of the orthosis device 100 is provided. In particular, the resistive element 138 was passively activated to less than approximately 20° of flexion. (See, e.g., Pollo, F. E. et al., Reduction of Medial Compartment Loads with Valgus Bracing of the Osteoarthritic Knee, Am. J. Sports Med., Vol. 30, pp. 414-421 (2002)). Curve (a) shows the load experienced by the knee 105 without implementation of the orthosis device 100, while curve (b) shows the load experienced by the knee 105 with the orthosis device 100. Area A represents the total reduction of the load experienced by the knee 105. As can be seen from FIG. 13, during the critical period between approximately 0% and approximately 55% of the gait cycle, the orthosis device 100 was found to substantially reduce the load experienced by the knee 105. For example, on average, the orthosis device 100 was found to reduce the load experienced by the knee 105 by approximately 200N. However, as discussed herein, it should be understood that the support provided by the exemplary orthosis device 100 may be customized based on the needs of a patient and, therefore, the resulting performance of the orthosis device 100 can vary.

It should be noted that the results presented above represent generalized outcomes of the orthosis device 100 and that several factors may influence the ultimate efficacy of the orthosis device 100. Although one function of the orthosis device 100 can be to reduce pain, the magnitude of the joint load may not be the sole factor in determining the outcome. For example, in general, there is not a one-to-one relationship between joint load and pain. In other words, a 50% load reduction generally does not generate a corresponding 50% pain reduction. Some studies have shown that an 11% load reduction in the knee 105 can provide a significant pain reduction. (See, e.g., Pollo, F. E. et al., Reduction of Medial Compartment Loads with Valgus Bracing of the Osteoarthritic Knee, Am. J. Sports Med., Vol. 30, pp. 414-421 (2002)). As a further example, osteoarthritis typically occurs in either the lateral or medial compartments of the knee 105. By varying the placement of the load bearing elements of the orthosis device 100 accordingly (e.g., medial vs. lateral), the effectiveness of the orthosis device 100 can be maximized. As discussed above, the two support elements in the orthosis device 100, e.g., the piston damper 124 and the resistive element 138, can be altered to yield a patient-specific outcome while being limited by safe biological forces. Thus, the exemplary orthosis device 100 discussed herein can reduce loads, moments, or both, in a knee 105 joint of a patient and provide muscle support to the patient, while allowing the patient to remain mobile. In addition, the exemplary orthosis device 100 can be adjusted or customized to provide a variable amount of support and stabilization to the patient based on the particular needs of the patient.

While exemplary embodiments have been described herein, it is expressly noted that these embodiments should not be construed as limiting, but rather that additions and modifications to what is expressly described herein also are included within the scope of the invention. Moreover, it is to be understood that the features of the various embodiments described herein are not mutually exclusive and can exist in various combinations and permutations, even if such combinations or permutations are not made express herein, without departing from the spirit and scope of the invention.

1. A knee orthosis device for providing support to a patient during a gait cycle, comprising:
a first support mechanism adapted to be positioned above a knee joint of the patient,
a second support mechanism adapted to be positioned below the knee joint of the patient, and
a central mechanism adapted to be positioned at or near the knee joint of the patient, the central mechanism mechanically connecting the first support mechanism and the second support mechanism,
wherein a link of the central mechanism translates relative to the second support mechanism during the gait cycle, and
wherein the second support mechanism includes a resistive element configured and dimensioned to receive at least a portion of the link therein, interaction between the resistive element and the link providing support to the patient.

2. The knee orthosis device according to claim 1, wherein the central mechanism is a four-bar linkage system comprising four links rotatably connected relative to each other.

3. The knee orthosis device according to claim 2, wherein motion of the four links of the central mechanism is adjustable based on at least one of a patient knee motion, a patient weight, a patient condition, a knee size, or a knee shape.

4. The knee orthosis device according to claim 1, wherein the central mechanism transfers forces between the first support mechanism and the second support mechanism.

5. The knee orthosis device according to claim 1, wherein the link is T-shaped and an extension of the link translates relative to the second support mechanism during the gait cycle.

6. The knee orthosis device according to claim 1, wherein the first support mechanism provides support to quadriceps muscles of the patent during a loading response phase of the gait cycle.

7. The knee orthosis device according to claim 1, wherein the first support mechanism comprises at least one of a piston damper, a rotary damper, a passive damper, or an elastic material.

8. The knee orthosis device according to claim 7, wherein the piston damper comprises a dampening structure therein, the dampening structure including at least one of a magnetorheological fluid or a spring.

9. The knee orthosis device according to claim 7, wherein the piston damper comprises an electromagnet, wherein varying a power supplied to the electromagnet varies a resistive force imparted by the piston damper.

10. The knee orthosis device according to claim 1, wherein the second support mechanism provides support during a stance phase of the gait cycle, and wherein the second support mechanism provides no support during a free swing phase of the gait cycle.

11. The knee orthosis device according to claim 1, wherein the second support mechanism comprises a calf connection.

12. The knee orthosis device according to claim 11, wherein the calf connection defines a planar surface configured to be positioned against a calf of the patient.

13. The knee orthosis device according to claim 11, wherein the calf connection comprises a groove track configured to slidably receive therein a complementary protrusion of the link of the central mechanism, the groove track and the protrusion forming a sliding joint.

14. The knee orthosis device according to claim 13, wherein the groove track is linear.

15. The knee orthosis device according to claim 1, wherein the link of the central mechanism comprises a coupler extending therefrom for interaction with the resistive element.

16. The knee orthosis device according to claim 15, wherein at least a portion of the coupler translates in and out of the resistive element as the link of the central mechanism translates relative to the second support mechanism during the gait cycle.

17. The knee orthosis device according to claim 15, wherein the coupler comprises a piston extending therefrom, the piston configured to be received by an aperture of the resistive element to align translation of the link of the central mechanism relative to the resistive element.

18. The knee orthosis device according to claim 1, wherein the resistive element comprises a resistive device disposed within a housing, the resistive device comprising at least one of a linear spring, a non-linear spring, a magnetic spring, or an elastic substrate.

19. A method of providing support to a patient during a gait cycle, the method comprising:
providing a knee orthosis device, the knee orthosis device including (i) a first support mechanism adapted to be positioned above a knee joint of the patient, (ii) a second support mechanism adapted to be positioned below the knee joint of the patient, the second support mechanism including a resistive element, and (iii) a central mechanism adapted to be positioned at or near the knee joint of the patient, the central mechanism mechanically connecting the first support mechanism and the second support mechanism, and the central mechanism including a link translatable relative to the second support mechanism during the gait cycle,
providing support during a loading response phase of the gait cycle with the first support mechanism, and
providing support during the stance phase of the gait cycle with the second support mechanism by translating at least a portion of the link into the resistive element of the second support element.

20. A knee orthosis device for providing support to a patient during a gait cycle, comprising:
a first support mechanism adapted to be positioned above a knee joint of the patient,
a second support mechanism adapted to be positioned below the knee joint of the patient, the second support mechanism including a calf connection, the calf connection including a groove track, and
a central mechanism adapted to be positioned at or near the knee joint of the patient, the central mechanism mechanically connecting the first support mechanism and the second support mechanism, and the central mechanism including a link translatable relative to the second support mechanism during the gait cycle,
wherein a complementary protrusion of the link of the central mechanism slidably translates within the groove track of the second support mechanism during the gait cycle.
